(19) United States
(12) Patent Application Publication
(10) Pub. No.: US 2020/0168343 A1
(43) Pub. Date: May 28, 2020

(54) DEVICE, SYSTEM, AND METHOD FOR CLASSIFICATION OF COGNITIVE BIAS IN MICROBLOGS RELATIVE TO HEALTHCARE-CENTRIC EVIDENCE

(71) Applicant: KONINKLIJKE PHILIPS N.V., EINDHOVEN (NL)

(72) Inventors: Vivek Varma Datla, Watertown, MA (US); Oladimeji Feyisetan Farri, Yorktown Heights, NY (US); Sheikh Sadid Al Hasan, Cambridge, MA (US); Kathy Mi Young Lee, Westford, MA (US); Junyi Liu, Windham, NH (US)

(73) Assignee: Koninklijke Philips N.V., Eindhoven (NL)

(21) Appl. No.: 16/080,041

(22) PCT Filed: Feb. 28, 2017
(86) PCT No.: PCT/IB2017/051154

§ 371 Date: Aug. 27, 2018

Related U.S. Application Data
(60) Provisional application No. 62/301,250, filed on Feb. 29, 2016.
(60) Provisional application No. 62/415,541, filed on Nov. 1, 2016.

(51) Int. Cl. G16H 80/00; G06Q 50/00; G16H 10/60; G16H 50/70; G06F 40/30

(52) U.S. Cl. CPC G16H 80/00; G06Q 50/01; G06F 40/30; G16H 50/70; G16H 10/60

(57) ABSTRACT

A device, system, and method classifies a cognitive bias in a microblog relative to healthcare-centric evidence. The method performed at a microblog server includes receiving a selection from a clinician, the selection indicating a health-related topic. The method includes determining evidence data of the health-related topic from validated information sources. The method includes receiving a microblog, the microblog associated with the health-related topic. The method includes determining a cognitive bias of the microblog based on the evidence data.

### BACKGROUND INFORMATION

A clinician may provide healthcare or health-related information to patients in person or through communications such as online communications. Even with the knowledge that the clinician may be skilled in a concentrated medical field, the clinician may still refer to external sources to aid in determining the proper healthcare or health-related information to provide to the patient. Despite the medical information available to the clinician, a patient may still agree/disagree or have a belief that coincides/contradicts the medical information in varying degrees of cognition. Thus, the clinician may be required to adjust the healthcare to be provided for this patient based on this cognition.

Due to the increase in microblogging and messaging within social networks, there is a continuous and voluminous stream of information on significantly impactful topics generated by and available to users. The information has many facets ranging from personal opinions to organizational news, subjective and relatively objective propositions, factual to fictional statements, and humorous to aggressive comments. Given the amount of available information, manually reviewing and comprehending the sentiments and cognitive biases hidden in large volumes of microblog text is a near-impossible task in addition to verifying the veracity based on existing facts and evidence. It is becoming increasingly difficult to automatically discover the cognitive bias of the author of a microblog post/message as such characteristics are typically implicit. Furthermore, the ability to comprehend the bases of ideas and opinions in microblogs based on available metadata or background information curated from users' interest profiles may be helpful to a few users but is largely insufficient due to the multi-faceted processes of human cognition and judgment that cannot be aptly captured by reviewing entries on demographics, vocation, or extracurricular activities.

The ubiquitous usage of microblogs and other social media for healthcare-centric discourse and debates also brings the challenge of tailoring explanations and interventions to match the cognitive biases of the authors of such messages to ensure the clinicians are well-informed, make the right clinical decision, and are better engaged in healthcare. For example, there is a relatively large amount of noise of varying lengths in messages on social media discussions. Classifying and filtering this data based on a cognitive-bias may be extremely useful to clinicians needing actionable information aside from a typical patient interaction in a hospital and/or clinic setting. Such a capability may enable the clinicians to promptly and effectively address the concerns and paranoia surrounding healthcare issues and/or services that have critical individual and public health significance. Such a capability may also enable further investigations into the underlying factors that lead to support, concerns or paranoia regarding healthcare breakthroughs, or adoption of health information technology. However, conventional approaches only focus on a sentiment analysis (e.g., identifying the sentiment, polarity, and opinion of a microblog message based on various linguistic features). Furthermore, the conventional approaches do not consider the veracity of the message relative to available evidence nor do the conventional approaches determine the cognitive (versus sentimental) bias of the authors.

### SUMMARY

The exemplary embodiments are directed to a method, comprising: at a microblog server: receiving a selection from a clinician, the selection indicating a health-related topic; determining evidence data of the health-related topic from validated information sources; receiving a microblog, the microblog associated with the health-related topic; and determining a cognitive bias of the microblog based on the evidence data.

The exemplary embodiments are directed to a microblog server, comprising: a transceiver communicating via a communications network, the transceiver configured to receive a selection from a clinician, the selection indicating a health-related topic, the transceiver configured to receive a microblog, the microblog associated with the health-related topic; a memory storing an executable program; and a processor that executes the executable program that causes the processor to perform operations, comprising, determining evidence data of the health-related topic from validated information sources, and determining a cognitive bias of the microblog based on the evidence data.

The exemplary embodiments are directed to a method, comprising: at a microblog server: receiving a selection from a clinician, the selection indicating a health-related topic; determining evidence data of the health-related topic from validated information sources; receiving a plurality of microblogs, each of the microblogs associated with the health-related topic; determining a respective cognitive bias for each of the microblogs based on the evidence data; and determining an overall cognitive bias for an audience associated with the microblogs.

### BRIEF DESCRIPTION OF THE DRAWINGS

FIG. 1 shows a system according to the exemplary embodiments.

FIG. 2 shows a microblog server of FIG. 1 according to the exemplary embodiments.

FIG. 3 shows a process flow to generate a weighted curated graph according to the exemplary embodiments.

FIG. 4 shows a method for determining a classification output of a microblog according to the exemplary embodiments.

### DETAILED DESCRIPTION

The exemplary embodiments may be further understood with reference to the following description and the related appended drawings, wherein like elements are provided with the same reference numerals. The exemplary embodiments are related to a device, a system, and a method for classification of a cognitive bias of an author of a microblog relative to healthcare-centric evidence. Specifically, the cognitive bias may be as to a particular stance the author has on a health-related topic. The exemplary embodiments are configured to automatically evaluate a cognitive bias of microblog posts on specific health-related topics of interest of a clinician such that the clinician may customize advocacy and interventions to match the unique cognitive characteristics of a target population or patient. In this manner, the clinician may provide a more efficient manner of care to the patient.

As will be described in detail below, the exemplary embodiments empower clinicians to synthesize a cognitive bias in microblog posts relative to facts and evidence in recognized and validated knowledge sources on a specific health-centric topic. The exemplary embodiments may classify the cognitive bias of real-time microblog posts into four classification outputs that reflect a baseline cognition with respect to a particular health-related issue for the author of the microblog. The exemplary embodiments further allow the discovery of a subjective perspective that may be otherwise implicit but which may be leveraged for personalized health education, interventions, and service delivery by a clinician.

It is noted that the exemplary embodiments are described with regard to a clinician and how the cognitive bias may be utilized by the clinician, particularly with respect to healthcare for a patient. However, the perspective of the clinician is only exemplary. The exemplary embodiments may be modified for use by any healthcare stakeholder (e.g., not necessarily a medical professional) who may utilize the cognitive bias for a variety of different reasons (e.g., polling purposes).

It is also noted that the exemplary embodiments are described with regard to health-related topics and microblogs. However, the use of the health-related topics is only exemplary. Those skilled in the art will understand that the exemplary embodiments may be modified accordingly to be used with any topic in which evidence is leveraged to identify a degree of a cognitive bias. Therefore, the health-related topics may represent any topic in which evidence in the topic may be leveraged. Furthermore, the use of microblogs is only exemplary. Those skilled in the art will understand that the exemplary embodiments may be modified accordingly to be used with any online or offline post by an author, whether micro or not. Therefore, the microblog may represent any manner in which a statement may be made.

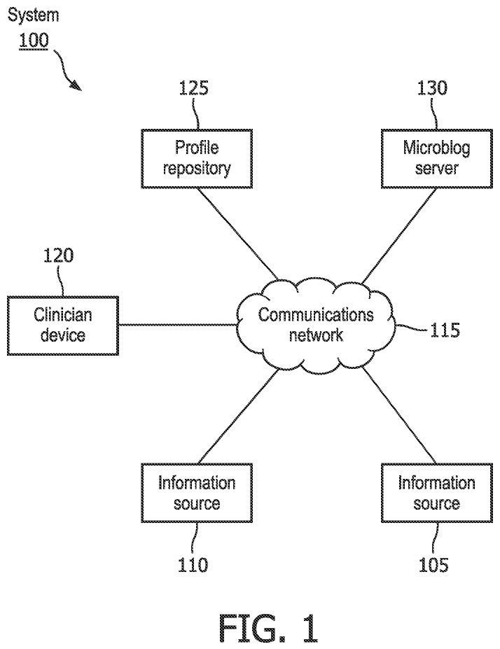

FIG. 1 shows a system 100 according to the exemplary embodiments. The system 100 relates to a communication between various components involved in determining a cognitive bias of a microblog based on available evidence for the particular health-related topic. Specifically, the system 100 may include a plurality of information sources 105, 110, a communications network 115, a clinician device 120, a profile repository 125, and a microblog server 130. As will be described in further detail below, the system 100 is configured to utilize the information sources 105, 110 which may be the source of a microblog as well as health-related evidence associated with a topic of the microblog.

The information sources 105, 110 may represent any source from which information is received. The information may be medical information/health-related evidence, online or digital information, microblogs, etc. For example, the information source 105 may include a repository for clinical reports in an electronic medical record (EMR). In another example, the information source 105 may include other medical-related data from medical journals, hospitals, medical libraries, etc. In a further example, the information source 110 may include online streams such as social media streams, health blogs, online news media, etc. For exemplary purposes, the information sources 105, 110 may provide any information that may be used as evidence for health-related topics. The information sources 105, 110 may also include microblog sites in which authors post microblogs.

It should be noted that the system 100 illustrating two information sources 105, 110 is only exemplary. The information sources 105, 110 may represent one or more information sources that are configured to provide the information to the other components of the system 100. In fact, the information sources 105, 110 may represent each individual item that may be available from a repository or source, the repository or source itself, a collection of repositories, etc.

The communications network 115 may be configured to communicatively connect the various components of the system 100 to exchange data. The communications network 115 may represent any single or plurality of networks used by the components of the system 100 to communicate with one another. For example, if the microblog server 130 is used at an administration site, the communications network 115 may include a private network in which the microblog server 130 may initially connect (e.g. a hospital network). The private network may connect to a network of an Internet Service Provider to connect to the Internet. Subsequently, through the Internet, a connection may be established to other electronic devices. It should be noted that the communications network 115 and all networks that may be included therein may be any type of network. For example, the communications network 110 may be a local area network (LAN), a wide area network (WAN), a virtual LAN (VLAN), a WiFi network, a HotSpot, a cellular network (e.g., 3G, 4G, Long Term Evolution (LTE), etc.), a cloud network, a wired form of these networks, a wireless form of these networks, a combined wired/wireless form of these networks, etc.

The clinician device 120 may represent any electronic device that is configured to perform the functionalities associated with a clinician. For example, the clinician device 120 may be a portable device such as a tablet, a laptop, etc. or a stationary device such as a desktop terminal. The clinician device 120 may include the necessary hardware, software, and/or firmware to perform the various operations associated with medical treatment. The clinician device 120 may also include the required connectivity hardware, software, and firmware (e.g., transceiver) to establish a connection with the communications network 115 to further establish a connection with the other components of the system 100. For example, the clinician device 120 may schedule appointments for patients using a calendar application, may track treatments or procedures of a patient, etc. In another example, the clinician device 120 may be used to post online content such as microblogs. In a further example and as will be described in further detail below, the clinician device 120 may receive notifications from the microblog server 130 regarding results of a microblog analysis for a health-related topic associated with the clinician.

The profile repository 125 may be a component that stores user profiles. Specifically, the profile repository 125 may store user profiles of clinicians. As will be described in further detail below, the microblog server 130 may generate user profiles that may be stored in the profile repository 125. If the profile repository 125 already has a user profile for a particular clinician, the relevance server 130 may query the profile repository 125 to retrieve the corresponding user profile.

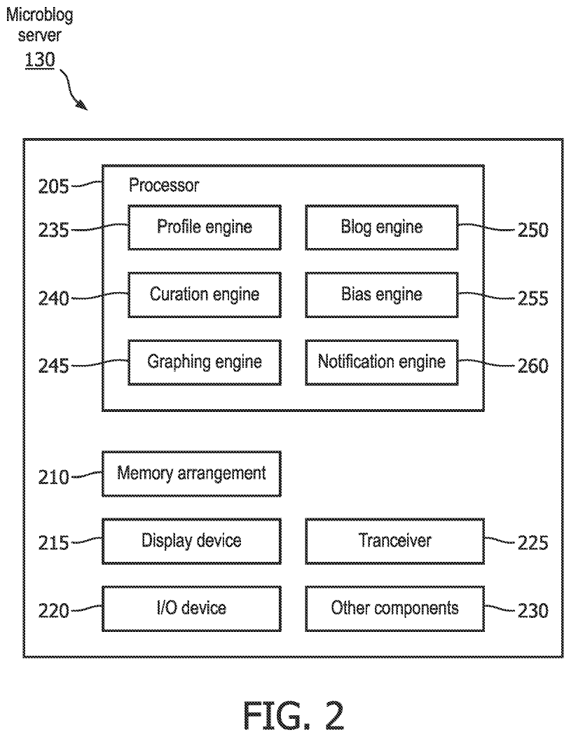

The microblog server 130 may be a component of the system 100 that performs functionalities associated with the features of the exemplary embodiments in which a cognitive bias is determined for an author of a microblog based on health-related evidence. FIG. 2 shows the microblog server 130 of FIG. 1 according to the exemplary embodiments. The microblog server 130 may provide various functionalities in determining the cognitive bias and notifying a clinician of the cognitive bias. Although the microblog server 130 is described as a network component (specifically a server), the microblog server 130 may be embodied in a variety of ways such as a portable device (e.g., a tablet, a smartphone, a laptop, etc.), a client stationary device (e.g., a desktop terminal), incorporated into the clinician device 120, incorporated into a website service, etc. The microblog server 130 may include a processor 205, a memory arrangement 210, a display device 215, an input and output (I/O) device 220, a transceiver 225, and other components 230 (e.g., an imager, an audio I/O device, a battery, a data acquisition device, ports to electrically connect the reporting server 130 to other electronic devices, etc.).

The processor 205 may be configured to execute a plurality of applications of the relevance server 125. As will be described in further detail below, the processor 205 may utilize a plurality of engines including a profile engine 235, a curation monitoring engine 240, a graphing engine 245, a blog engine 250, a bias engine 255, and a notification engine 260. The profile engine 235 may determine interest profiles of a clinician through various queries. The curation engine 240 may identify relevant knowledge and metadata based on the interest profiles of the clinician. The graphing engine 245 may normalize and rank available evidence as well as identify semantic matching of the microblog with the evidence to generate a three-dimensional nodal graph. The blog engine 250 may analyze, clean, and normalize a microblog or microblog related data. The bias engine 255 may identify the cognitive bias of the microblog based on the other available information from the other engines. The notification engine 260 may generate notifications for the clinician of a determined cognitive bias.

It should be noted that the above noted applications and engines each being an application (e.g., a program) executed by the processor 205 is only exemplary. The functionality associated with the applications may also be represented as components of one or more multifunctional programs, a separate incorporated component of the microblog server 130 or may be a modular component coupled to the microblog server 130, e.g., an integrated circuit with or without firmware.

The memory 210 may be a hardware component configured to store data related to operations performed by the microblog server 130. Specifically, the memory 210 may store data related to the various engines 235-260 such as the user profile of the clinician and the data from the information sources 105, 110. The display device 215 may be a hardware component configured to show data to a user while the I/O device 220 may be a hardware component that enables the user to enter inputs. For example, an administrator of the microblog server 130 may maintain and update the functionalities of the microblog server 130 through user interfaces shown on the display device 215 with inputs entered with the I/O device 220. It should be noted that the display device 215 and the I/O device 220 may be separate components or integrated together such as a touchscreen. The transceiver 225 may be a hardware component configured to transmit and/or receive data via the communications network 110.

According to the exemplary embodiments, the microblog server 125 may perform various different operations to determine the cognitive bias of a microblog. Initially, as described above, the profile engine 235 may determine interest profiles of a clinician through various queries. When a clinician selects to utilize the services of the microblog server 130 (e.g., on a website hosted by the microblog server 130 or executing an application that connects to the microblog server 130), the clinician may be provided a form or be requested for information to be entered. Specifically, the clinician may select a health-related topic of interest (e.g., from a list of pre-generated health-related topics) and provide corresponding details in a concise user interest profile. The health-related topic of interest may range from general topics (e.g., heart disease, cancer, neural conditions, etc.) or may be more specific (e.g., coronary artery disease, lung cancer, autism, etc.). The clinician may enter the details as an unstructured text in a query interface with the microblog server 130.

It is noted that the profile engine 235 may be utilized each time the clinician selects a unique health-related topic of interest that was previously not selected. For example, when the clinician has selected the health-related topic of interest and has entered the corresponding details, the profile engine 235 may generate the interest profile for the clinician related to the selected health-related topic (e.g., which may be stored in the profile repository 125). Each time the clinician selects a new, unique health-related topic, the user profile of the clinician may be updated with an interest profile corresponding to the selected health-related topic.

It is also noted that the profile engine 235 generating the user profile and/or the interest profiles based on manually provided inputs is only exemplary. According to another exemplary embodiment, the profile engine 235 may be configured to automatically identify a topic of interest for a clinician as well as determine the interest profile for the identified topic of interest using various operations that gather information of the clinician and analyze this information. For example, the profile engine 235 may utilize monitor or receive information of the clinician from the information sources 105, 110 that provide insight as to the topic of interest and the interest profile.

As described above, the curation engine 240 may identify relevant knowledge and metadata based on the interest profiles of the clinician. The curation engine 240 may parse the user profile and/or the interest profile for the selected health-related topic to build a curated knowledge database by assimilating existing knowledge on the selected health-related topic from validated sources of evidence. For example, the information sources 105, 110 may include validated online sources, libraries, published biomedical literature, etc. Accordingly, the selected health-related topic may have all relevant and validated evidence associated therewith.

As described above, the graphing engine 245 may normalize and rank available evidence as well as identify semantic matching of the microblog with the evidence to generate a three-dimensional nodal graph. Initially, it is noted that the graphing engine 245 and the use of a graph is only exemplary. The exemplary embodiments may utilize any mechanism in which the information from the curation engine 240 is to be organized for use with the subsequent aspects of the exemplary embodiments. Furthermore, it is noted that the use of the three-dimensional nodal graph is only exemplary and, again, any graph or representation may be used.

The graphing engine 245 may convert the information from the curation engine 240 into a knowledge graph-like structure such as the three-dimensional nodal graph based on various relations. Specifically, the relations may be an agent-action-patient (AAP) relationship that reflects the semantic roles identified by a semantic role labelling operation. In an illustrative example, the AAP relationship for the sentence “User threw a ball at the window.” may entail “user” to be the agent, “threw” to be the action, “ball” to be the patient, and “window” to be the benefactor (entity indirectly affected by the action). The AAP relationships determined from the information of the curation engine 240 may be ingested by a semantic roles-to-graph operation for conversion into the three-dimensional nodal graph. As will be utilized herein, the resulting three-dimensional nodal graph from the curated information will be referred to as the “curated graph”.

It is noted that the three-dimensional nodal graph utilizing the AAP relationship is only exemplary. According to another exemplary embodiment, the graphing engine 245 may utilize other types of relations such as a phrase-word-phrase (PWP) relationship. Those skilled in the art will understand that the PWP relationship may be utilized for other purposes such as representing complex relations.

The graphing engine 245 may further have the semantic relations weighted based on ranked keywords present in the evidence gathered by the curation engine 240. For example, the graphing engine 245 may utilize a rake functionality in which the ranked keywords are identified with existing keyword extraction libraries. Accordingly, the three-dimensional nodal graph generated by the graphing engine 245 may be a weighted graph representing causative relations identified in the curated information by the curation engine 240. Thus, a weighted curated graph may be generated.

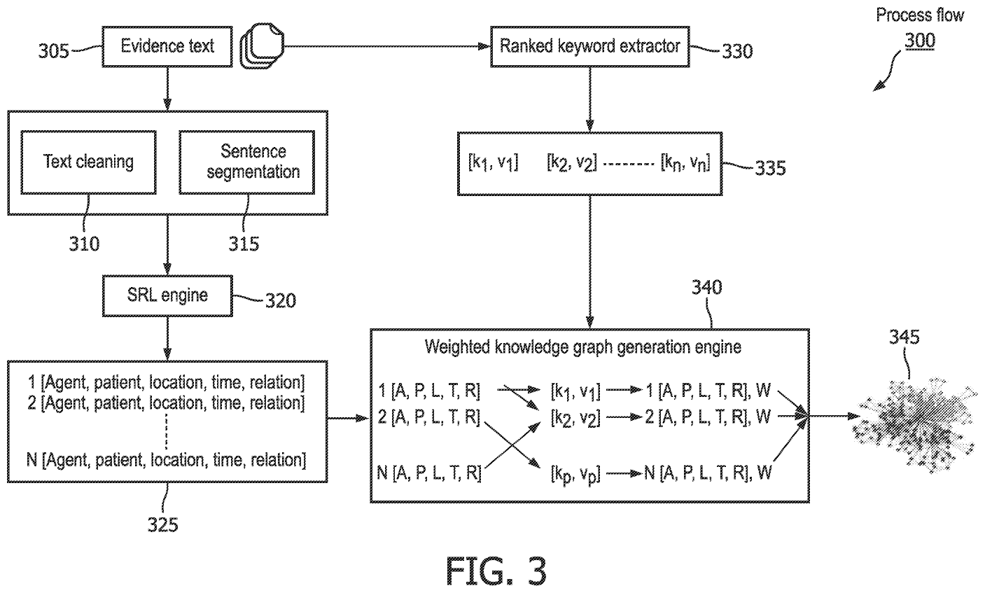

FIG. 3 shows a process flow 300 to generate a weighted curated graph 345 according to the exemplary embodiments. The process flow 300 illustrates one particular manner in which the weighted curated graph 345 may be generated. Specifically, the process flow 300 may relate to the operations performed by the curation engine 240 and the graphing engine 245. It is noted that the process flow 300 is only exemplary and the exemplary embodiments may utilize other mechanisms or modified process flows to generate the weighted curated graph 345. As described above, the exemplary embodiments may utilize a first portion in which a curated graph is generated and a second portion in which a weighting is determined, the first and second portions being combined to generate the weighted curated graph. As will be described in further detail below, the first portion may include a plurality of processes 305-325 while the second portion may include a plurality of processes 305, 330, 335. The first and second portions may be combined for a process 340 to generate the weighted curated graph 345.

The process flow 300 may include a process 305 in which text associated with evidence is received. Specifically, the evidence text that may be identified by the user and/or online sources may be curated using a semantic role labeling (SRL) operation as depicted as process 320. Prior to the SRL operation in process 320, the process flow 300 may include a process 310 in which a text cleaning operation is performed and a process 315 in which a sentence segmentation operation is performed. Accordingly, the text from the process 305 may be normalized for the process 320. The output of the SRL operation may be a set of semantic relations with respect to the verbs identified in the phrases of the text. For example, the relations may include the AAP relationship described above. Thus, in the process 325, the semantic relations may be converted to resource description format (RDF) triples that identify an agent, a patient, a benefactor, a location, a time, etc. The agent may correspond to a subject performing an action while and the patient may refer to a recipient of the action. The verb may identify the action in this relationship. In this manner, a plurality of relations may be established for the text from the process 305.

In the second portion, the triples or relations may be weighted by having a measure such as term frequency/inverse document frequency from the key word extraction operation described above. For example, in process 330, the weights provided may be tuned with respect to the domain and interest of the user. The weights may indicate the relative importance of the words with respect to the curated text. In the process 335, the key word weights may be encoded into the semantic triples and each RDF triple may have a cumulative as well as independent weight score. As shown, the pairing may be for a word (k) to a weight of the word (v).

Through combination of the above portions, the process 340 may entail saving the relations into a weighted curated graph 345 such as a three-dimensional nodal graph. Specifically, the triples may utilize the agent (A), the patient (P), the location (L), the time (T), and the relation (R) to which the cumulative weight (W) is associated. Given new microblog text, the semantic roles may be extracted from the text to identify the similar nodes in the weighted curated graph based on semantic similarity operations, deep learning based models, other machine learning models, etc. such that the text is ranked corresponding to the curated knowledge.

As described above, the blog engine 250 may analyze, clean, and normalize a microblog or microblog related data. Initially, the blog engine 250 may receive a microblog or a plurality of microblogs from the information sources 105, 110. The microblogs may relate to any of a variety of topics, in particular health-related topics. However, the microblogs may be unaware as to the topic of the microblog until further analysis is performed. Accordingly, the blog engine 250 may determine when new microblogs are available and perform the operations herein.

The blog engine 250 may normalize the microblog from various sources. For example, the microblog may be a stand-alone message posted by an author on an online site. In another example, the microblog may be extracted from a social media colloquy. The blog engine 250 may also perform this operation in real-time as the microblog becomes available. In light of the language that is utilized in microblogs, the blog engine 250 may utilize a natural language processing (NLP) functionality that analyzes the syntax and extracts semantic elements and keywords from the microblog. Specifically, the NLP functionality may utilize various different operations. In a first example, a generic rule-based operation may be used for sentence boundary detection such as periods, question marks, exclamation marks, etc. In a second example, a language model operation may be used for part-of-speech tagging. In a third example, a machine learning classifier operation that is trained on comprehensive English language corpora may be used for phrase chunking (e.g., to break down the grammatical statements into chunks representing noun phrases, adjective phrases, verb phrases, etc.). In a fourth example, a dictionary-driven operation may be used to map the chunks and acronyms to recognized English keywords. In a fifth example, a disambiguation operation may be used to disambiguate the sense of an extracted word using the contextual elements in the text (e.g., to determine that ‘bank’ in a narrative with emphasis on ‘economy’ refers to the financial institution, not the ‘boundaries of a river or lake’).

It is noted that the microblog may be analyzed for further aspects. For example, the blog engine 250 may include a further functionality or sub-engine such that the sentiment and/or the opinion of the microblog may be analyzed. Specifically, the normalized microblog may be analyzed for sentiment such as positive, negative, or neutral. The normalized microblog may also be analyzed for subjectivity as to whether the opinion is subjective or objective. The normalized microblog may further be analyzed using the above described AAP relationship. Various tools for sentiment analysis and opinion mining may be leveraged to extract the polarity and subjectivity of the microblog, respectively. Those skilled in the art will understand that measuring the subjectivity may aid in identifying how opinionated a microblog is.

The information from the blog engine 250 may further be provided to the graphing engine 245 such that a three-dimensional nodal graph for the microblog may also be generated. As noted above, the microblog may also be analyzed with the AAP relationship. Thus, utilizing a substantially similar operation described above for the curated information from the curation engine 240, the normalized microblog and the information thereof may be used to generate a three-dimensional nodal graph. As the three-dimensional nodal graph relates only to the microblog, there is a higher likelihood that the three-dimensional nodal graph is far less complex than the three-dimensional nodal graph of the selected health-related topic from the clinician. However, it is noted that the graphing engine 245 may utilize a further operation to expand the vocabulary of the relations in the microblog by using a deep learning-based neural word/phrase embedding operation that identifies semantically similar words. As will be utilized herein, the resulting three-dimensional nodal graph from the microblog will be referred to as the “microblog graph”.

As described above, the bias engine 255 may identify the cognitive bias of the microblog based on the other available information from the other engines. Specifically, the bias engine 255 may receive the weighted curated graph for the selected health-related topic from the clinician using the information from the profile engine 235 and the curation engine 240 as well as the microblog graph for the microblog using the information from the blog engine 250. The bias engine 255 may utilize the weighted curated graph and the microblog graph to perform a ‘fuzzy graph walk’ operation where the weighted curated graph is referenced with the microblog graph. For example, the fuzzy graph walk may initially be used to determine whether the microblog has any relevance to the selected health-related topic for the clinician. The expanded vocabulary based on neural embeddings for the microblog graph may increase the recall for a match with the evidence in the weighted curated graph during the graph walk.

Based on the above operation, the bias engine 255 may generate a “fuzzy-match” score for the microblog based on the partial matches on the weighted evidence in source of the evidence, sentiment expressed in the microblog, and support for that sentiment in the weighted curated graph. Specifically, the microblog may be classified into one of four categories of cognitive bias: (1) nonchalant, (2) proponent, (3) concerned, and (4) paranoid. These categories may be identified based on the fuzzy-match score, sentiment, and opinion scores. The fuzzy-match score, the sentiment, and the opinion scores may be utilized to generate a cognitive value. Each of the categories may have a cognitive range such that the cognitive value may indicate which of the cognitive biases is determined. For example, “nonchalant” may be a range from zero to a first threshold; “proponent” may be a range from the first threshold to a second threshold; “concerned” may be a range from the second threshold to a third threshold; and “paranoid may be a range above the third threshold.

The notification engine 260 may generate notifications for the clinician based on the result generated by the bias engine 255. For example, the clinician may be alerted on the cognitive bias of a microblog in real-time to facilitate a prompt and targeted communication/intervention to the author. Specifically, the communication may be in terms of education or care services (e.g., counseling) to ensure that the author of the microblog is better informed to make the correct health-related decision and be motivated to take active steps leading to desired health outcomes.

It is noted that the exemplary embodiments may be used in a variety of different implementations and provide results used for a variety of different reasons. In a first example, the clinician may utilize the features of the exemplary embodiments in a targeted patient approach. Specifically, the microblog that is analyzed may be authored by a patient of the clinician. Thus, the targeted patient approach may relate to when the microblog of the patient is identified and the cognitive bias that is identified may be provided to the clinician. In this manner, a notification for the particular patient may be provided to the clinician and the clinician may thereby more appropriately cater the healthcare for the patient in light of this knowledge. For example, if the microblog indicates a nonchalant cognitive bias, the clinician may understand that the patient has mentioned a particular statement for a selected health-related topic but is not strongly suggestive to a particular direction given the known validated evidence in this health-related topic. In another example, if the microblog indicates a paranoid cognitive bias, the clinician may determine that further healthcare related to the selected health-related topic may warrant a particular manner of providing healthcare to accommodate the cognitive bias of the patient.

In a second example, the clinician may utilize the features of the exemplary embodiments in a targeted bias approach. Specifically, the clinician may receive notifications for authors of microblogs who have a cognitive bias that is of at least a predetermined cognitive bias. For example, using the four types of biases described above, the clinician may receive notifications for microblogs that have at least a concerned or paranoid cognitive bias. The clinician may provide information to these authors such as to assuage any fears that the authors may have.

In a third example, the clinician may utilize the features of the exemplary embodiments in a general audience approach. Specifically, the clinician may receive notifications that provide an overview of cognitive biases for a selected health-related topic. For example, using the four cognitive biases described above, a selected health-related topic may indicate a percentage of each cognitive bias based on microblogs that are identified to pertain to the selected health-related topic. In this manner, the clinician may be aware of a general cognitive bias that a general audience has for the selected health-related topic.

In a fourth example, an entity may utilize the features of the exemplary embodiments in a polling approach. Specifically, the polling approach may provide the cognitive bias or an overview of the cognitive bias (e.g., as was performed for the general audience approach) may be performed for various different parameters of the audience. For example, the cognitive bias may be measured for a selected health-related topic based on a geographic location of the audience. In another example, the cognitive bias may be measured for a selected health-related topic based on an age group of the audience. Other examples may include a nationality, a historical condition, etc. In this manner, general knowledge for cognitive bias may be provided for various topics for various parameters.

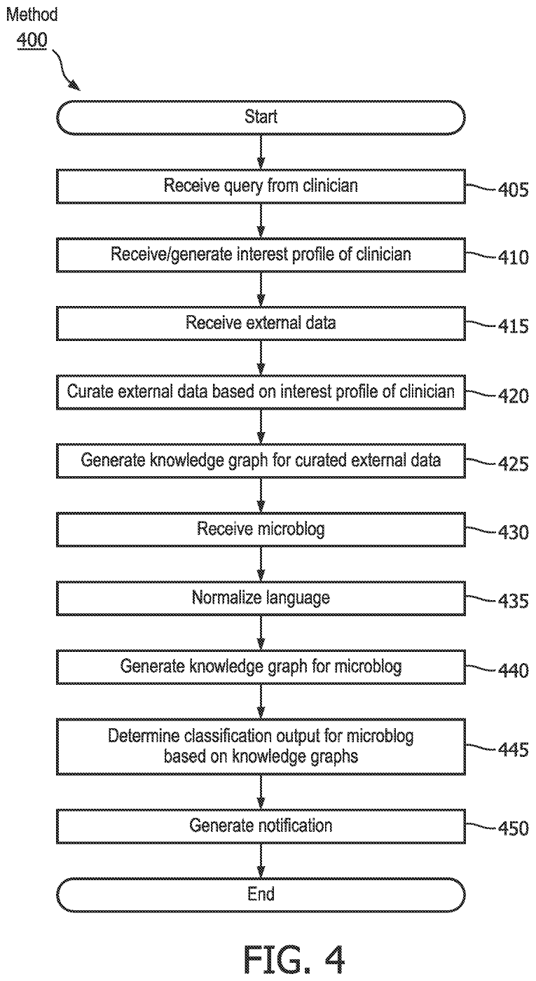

FIG. 4 shows a method 400 for determining a classification output of a microblog according to the exemplary embodiments. Specifically, the method 400 may relate to the mechanism of the exemplary embodiments in which the classification output is a cognitive bias associated with a microblog based on validated evidence in the health-related topic of the microblog. Accordingly, the method 400 will be described from the perspective of the microblog server 130. The method 400 will also be described with regard to the system 100 of FIG. 1 and the plurality of engines 235-260 of the microblog server 130 of FIG. 2.

In step 405, the microblog server 130 via the profile engine 235 receives a query from a clinician. As described above, the clinician may select a health-related topic and provide details associated with the health-related topic. The clinician may enter the information using a variety of different manners such as unstructured text. In step 410, the microblog server 130 via the profile engine 235 generates an interest profile of the clinician based on the topic and details. For example, the microblog server 130 may parse the text and utilize NPL operations. The interest profile may be specific to the selected health-related topic and associated with a user profile of the clinician. It is noted that if the interest profile and/or the user profile already exists and is stored in the profile repository 125, the microblog server 130 may retrieve the profiles.

In step 415, the microblog server 130 via the curation engine 240 may receive external data. Specifically, the external data may be evidence from validated various sources (e.g., information sources 105, 110) that relate to the selected health-related topic. In step 420, the microblog server 130 via the curation engine 240 may curate the external data based on the interest profile of the clinician related to the selected health-related topic. In step 425, the microblog server 130 via the graphing engine 245 may generate a curated graph. As described above, the curated graph may be a three-dimensional nodal graph in which an AAP relationship is determined for the curated information. The curated graph may also be weighted based on ranked keywords in the external data to generate a weighted curated graph.

In step 430, the microblog server 130 via the blog engine 250 may receive a microblog. For example, via the information sources 105, 110, the microblog server 130 may determine when a new microblog is available. When the new microblog is identified, in step 435, the microblog server 130 via the blog engine 250 may normalize the language. As described above, a microblog may utilize unconventional grammar, structure, and symbols. Thus, the microblog server 130 may utilize various different operations to normalize the language of the microblog. In step 440, the microblog server 130 via the graphing engine 245 may generate a microblog graph in a substantially similar manner as the weighted curated graph.

In step 445, the microblog server 130 via the bias engine 255 may determine a classification output for the microblog based on the weighted curated graph and the microblog graph. For example, a fuzzy graph walk operation may be utilized where the weighted curated graph and the microblog graph are referenced to one another. Subsequently, the microblog server 130 may determine the cognitive bias of the microblog. For example, the cognitive bias may be nonchalant, proponent, concerned, or paranoid. Thus, in step 450, the microblog server 130 via the notification engine 260 may generate a notification corresponding to the determined cognitive bias.

The exemplary embodiments provide a device, system, and method of determining a cognitive bias of a microblog based on evidence from validated sources of a health-related topic of the microblog. The mechanism according to the exemplary embodiments may receive information from a clinician to determine a selected health-related topic in which the evidence from the validated sources are curated. The microblog may be associated with the selected health-related topic such that microblog and the evidence is used to determine the cognitive bias of the microblog.

Those skilled in the art will understand that the above-described exemplary embodiments may be implemented in any suitable software or hardware configuration or combination thereof. An exemplary hardware platform for implementing the exemplary embodiments may include, for example, an Intel x86 based platform with compatible operating system, a Windows platform, a Mac platform and MAC OS, a mobile device having an operating system such as iOS, Android, etc. In a further example, the exemplary embodiments of the above described method may be embodied as a computer program product containing lines of code stored on a computer readable storage medium that may be executed on a processor or microprocessor. The storage medium may be, for example, a local or remote data repository compatible or formatted for use with the above noted operating systems using any storage operation.

It will be apparent to those skilled in the art that various modifications may be made in the present disclosure, without departing from the spirit or the scope of the disclosure. Thus, it is intended that the present disclosure cover modifications and variations of this disclosure provided they come within the scope of the appended claims and their equivalent.

1. A method, comprising:
at a microblog server:
receiving a selection from a clinician, the selection indicating a health-related topic;
determining evidence data of the health-related topic from validated information sources;
receiving a microblog, the microblog associated with the health-related topic; and
determining a cognitive bias of the microblog based on the evidence data.

2. The method of claim 1, wherein the selection includes detail information for the health-related topic, an interest profile for the clinician being generated based on the health-related topic and the detail information.

3. The method of claim 1, further comprising:
generating a curated graph based on the evidence data and the selection, the curated graph indicative of curated semantic relations of the evidence data and the selection.

4. The method of claim 3, wherein the curated graph is a three-dimensional nodal graph.

5. The method of claim 3, wherein the semantic relations are between an agent, an action, and a patient.

6. The method of claim 3, further comprising:
determining weights of the semantic relations based on extraction libraries; and
generating a weighted curated graph based on the curated graph and the weights.

7. The method of claim 1, further comprising:
generating a microblog graph based on the microblog, the microblog indicative of microblog semantic relations of the microblog.

8. The method of claim 7, wherein the microblog semantic relations utilize expanded vocabulary terms based on a deep learning-based neural word and phrase embedding operation to identify semantically similar words.

9. The method of claim 1, further comprising:
determining at least one of a sentiment and an opinion of the microblog to identify a polarity and a subjectivity, respectively, associated with the microblog.

10. The method of claim 1, wherein the cognitive bias is one of nonchalant, proponent, concerned, and paranoid.

11. A microblog server, comprising:
a transceiver communicating via a communications network, the transceiver configured to receive a selection from a clinician, the selection indicating a health-related topic, the transceiver configured to receive a microblog, the microblog associated with the health-related topic;
a memory storing an executable program; and
a processor that executes the executable program that causes the processor to perform operations, comprising,
determining evidence data of the health-related topic from validated information sources, and
determining a cognitive bias of the microblog based on the evidence data.

12. The microblog server of claim 11, wherein the selection includes detail information for the health-related topic, an interest profile for the clinician being generated based on the health-related topic and the detail information.

13. The microblog server of claim 11, wherein the processor further generates a curated graph based on the evidence data and the selection, the curated graph indicative of curated semantic relations of the evidence data and the selection.

14. The microblog server of claim 13, wherein the curated graph is a three-dimensional nodal graph.

15. (canceled)

16. (canceled)

17. (canceled)

18. (canceled)

19. (canceled)

20. A method, comprising:
at a microblog server:
receiving a selection from a clinician, the selection indicating a health-related topic;
determining evidence data of the health-related topic from validated information sources;
receiving a plurality of microblogs, each of the microblogs associated with the health-related topic;
determining a respective cognitive bias for each of the microblogs based on the evidence data; and
determining an overall cognitive bias for an audience associated with the microblogs.
